(19) United States
(12) Patent Application Publication
(10) Pub. No.: US 2026/0345369 A1
(43) Pub. Date: Nov. 5, 2026

(54) URINE COLLECTION AND MONITORING SYSTEM

(71) Applicant: Robert Boldt, Farmington, NY (US)

(72) Inventor: Robert Boldt, Farmington, NY (US)

(21) Appl. No.: 19/180,240

(22) Filed: Apr. 16, 2025

Related U.S. Application Data
(60) Provisional application No. 63/648,867, filed on May 17, 2024.

(51) Int. Cl. A61F 5/44; A61F 5/451

(52) U.S. Cl. CPC A61F 5/4405; A61F 5/4408; A61F 5/451

(57) ABSTRACT

The present invention is a urine collection bag mounting system, comprising: a frame member, a collection bag having an inlet and an outlet secured to the frame member, a tube connected to the inlet of the collection bag, and a strap connected to the frame member and the collection bag, wherein the strap is secured around a wearer's leg.

### CROSS-REFERENCE TO RELATED APPLICATIONS

This application is a continuation-in-part (and claims the benefit of priority under 35 USC 120) of U.S. application No. 63/648,867 filed May 17, 2024, now pending. The disclosure of the prior applications is considered part of (and is incorporated by reference in) the disclosure of this application.

### BACKGROUND OF THE INVENTION

The present invention relates to a urine collection system, and more particularly to a system for mounting a collection bag to the wearer's leg in a more secure and convenient manner.

Currently, patients dealing with urinary incontinence and that are catheter users tend to fall into one the following categories: 1) patients with indwelling or Foley catheters who rely on some type of drainage/urine collection bag; or 2) male patients who wear an external or condom catheter (or sheath catheter) that also require a urine drainage bag or collection system. Patients, male or female, who have to rely on some form of collection or drainage bag are faced with a number of challenges such as portability of such a collection bag without creating wardrobe issues, freedom of movement and ease of drainage of the collection bag, especially in public restrooms, just to name a few.

Current designs for urine collection fall into two broad categories; high-capacity bags (1500-2500 ml) with lengthy connecting tubes to be left at bed-side for bed-ridden/night-time use or low-capacity bags (400-700 ml) with much shorter tubes to be worn on the patient's body, for day-time/active wear. For some patients, it is customary to switch between these two modes each day.

The night-time bag is a satisfactory solution for bed-ridden patients who are essentially immobile, however the tubing running from the catheter to the stationary bag can inhibit an active person's normal movements during sleep and result in poor quality of rest.

Day-time setups typically use much shorter tubes and are attached to the wearer in one of three general manners; undergarments, sleeves, or straps. Undergarments typically consist of elastic material which includes pockets for the bags and sometimes sewn channels for the location of the connecting tubes. These undergarments locate the bags on the lap, the hip or high on the outer thigh. This placement is problematic for a number of reasons. It causes unsightly bulges in all but the loosest of clothing and it positions the bag in jeopardy of being squeezed and/or burst by anything more than a minor bump with furniture, appliances, etc., particularly by over-exuberant pets or children. Sleeves and straps are both offered in designs that can be attached to either the thigh or calf. Sleeves are offered ranging from simple knitted sock-like sleeves to heavy fabric with vertical straps to suspend them from a belt or waistband rather like a garter belt and various models support the bag on either the thigh or calf. The knitted sleeves are so insubstantial as to offer little or no support for even an only partially filled bag. The fabric sleeves are bulky and involve a more elaborate installation and fitting process. The straps generally supplied along with bags are typically of ½″-⅝″ width and very light duty. They sag under the weight of the bags and tend to slide down the leg as weight is added and/or activity increases.

Further problems relate to the emptying of the bag of urine. Either the user has to partially undress in order to empty the bag, or he has to wear trousers loosely-fitting enough to allow a user to roll up the trouser leg to get at it. Both methods are cumbersome. and likely embarrassing. Another disadvantage is that there is insufficient support for the storage or collection bag beyond the straps fitted around the leg, which presently are both tight and uncomfortable yet insecure (i.e., the straps tended to restrict blood circulation yet could not always prevent the storage unit from slipping down the leg). Also, the storage bag prevents the user from wearing ordinary, closely-fitted street clothes, because the collection bag outline could show through and because the trouser leg could not be rolled up high enough to allow the user to access a bag on the thigh. Current designs for calf-mounted bags do not support even empty bags at a height which is high enough that the drain valves are concealed by the bottom of the pant leg, thereby causing embarrassment due to advertising the presence of a collection bag.

An internal valve is located in the input port of virtually all bags of current manufacture. This valve is a check valve and serves to prevent the backflow of urine from the bag, back into the tube. Some manufacturers utilize a hard plastic mechanical valve for this function, but most use a much simpler and less costly design commonly called a “flutter valve”. The flutter valve consists of nothing more than a flattened section of thin-walled plastic tubing. Flowing from one direction expands the tubing and passes through the device into the bag, while flowing in the opposite direction is blocked by the collapsed section. The difficulty with this design is that if the flutter valve is not maintained in linear alignment it can be kinked and prevent flow in the desired direction. This condition can occur if the top portion of the bag becomes folded or if the input port becomes twisted relative to the bag itself.

Therefore, there is a need for a urine collection and drainage device system that is simple to use, maintains a stable location during common activities, and facilitates ease of drainage for the mobile and active patient. The present invention mitigates all of the above-mentioned difficulties and due to the greatly enhanced stability, the assembly is suitable for uninterrupted use, both day and night-time.

### SUMMARY

In a first embodiment, the present invention is a urine collection bag mounting system, comprising: a frame member; a strap, wherein the strap is attached to the frame member; and a collection bag secured to the frame.

In a second embodiment, the present invention is a urine collection bag mounting system, comprising: a frame member; a collection bag having an inlet and an outlet secured to the frame member; a tube connected to the inlet of the collection bag; and a strap connected to the frame member, wherein the strap is secured around a wearer's leg.

In a third embodiment, the present invention is a urine collection bag mounting system, comprising: A multi-layered frame member; a collection bag having an inlet and an outlet secured to the frame member; a sensor integrated into the collection bag; a tube connected to the inlet of the collection bag; and a strap connected to the frame member and the collection bag, wherein the strap is secured around a wearer's leg.

### BRIEF DESCRIPTION OF THE DRAWINGS

FIG. 1A depicts a front view of a urine collection device frame, in accordance with one embodiment of the present invention.

FIG. 1B depicts a front view of a urine collection device frame, in accordance with one embodiment of the present invention.

FIG. 2A depicts an isometric view of the urine collection device frame, in accordance with one embodiment of the present invention.

FIG. 2B depicts an isometric view of the urine collection device frame, in accordance with one embodiment of the present invention.

FIG. 3 depicts a front view of a drainage bag, in accordance with one embodiment of the present invention.

FIG. 4 depicts a rear view of a urine collection device assembly, in accordance with one embodiment of the present invention.

FIG. 5 depicts a rear view of the urine collection device assembly, in accordance with one embodiment of the present invention.

FIG. 6 depicts a rear view of the urine collection device assembly, in accordance with one embodiment of the present invention.

FIG. 7 depicts a front view of the urine collection device assembly, in accordance with one embodiment of the present invention.

FIG. 8 depicts a view of the urine collection device assembly in use, in accordance with one embodiment of the present invention.

### DETAILED DESCRIPTION OF THE INVENTION

The present invention provides a device that maintains alignment of the input valve and allows a secure fit to the wearer's calf that can fully conceal the drainage bag, allows for the wearer to be active and have little to no catching or snagging of the collection bag on their clothing, while also providing low effort and simple means to empty the collection bag without having to remove any articles of clothing.

The following below are more detailed descriptions of various embodiments of the invention described herein. In particular, the various embodiments disclosed herein describe a urine collection and drainage device. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of the ordinary skills in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

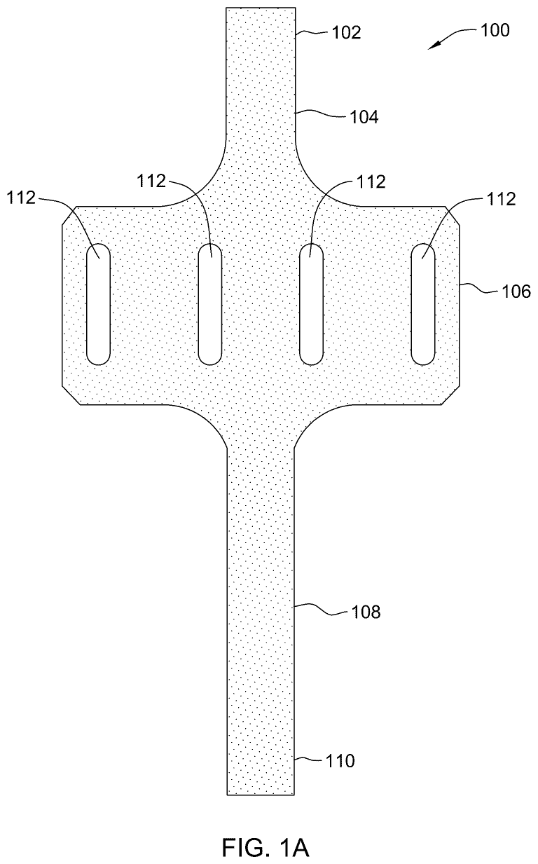

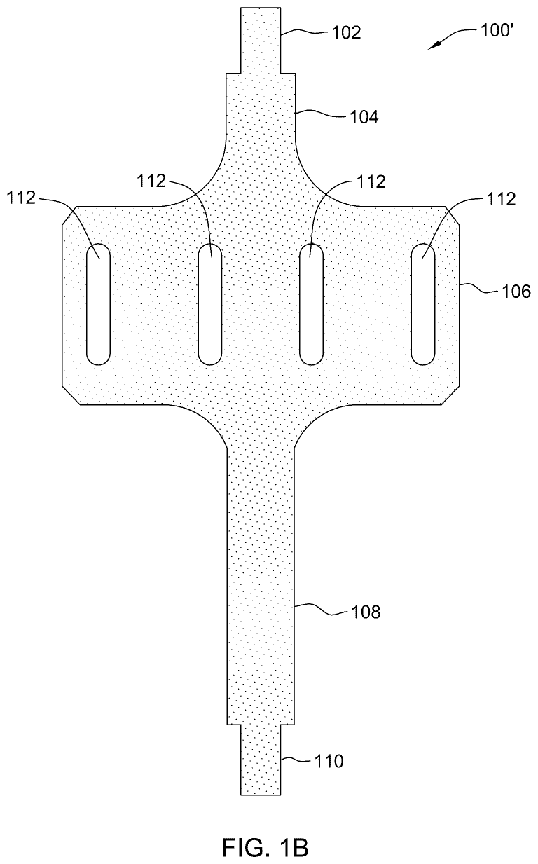

FIGS. 1A And 1B depict front views of a urine collection device frame 100, in accordance with one embodiment of the present invention. The urine collection device frame 100 (hereinafter “frame”) has a cruciform structure that is roughly the length of a drainage bag 300. The frame 100 has a top portion 104, a bottom portion 108, and a middle portion 106 that has a series of slots 112. In some embodiments (FIG. 1B), the top portion 104 has a notch 102 at a top end and the bottom portion 108 has a notch 110 at the bottom end. In the depicted embodiment there are four (4) slots 112. In additional embodiments there may be more or fewer slots 112. The middle portion 106 is a predetermined height and a predetermined length based on the drainage bag 300 design. The top portion 104 and the bottom portion 108 are of predetermined length and width based on the drainage bag 300 design, size, and the type of valve which is used. The top portion 104 and the bottom portion 108 are of predetermined lengths and widths based on the drainage bag 300 size and the required strength of the frame 100 to support the drainage bag 300 and secure the urine collection device to a wearer's leg.

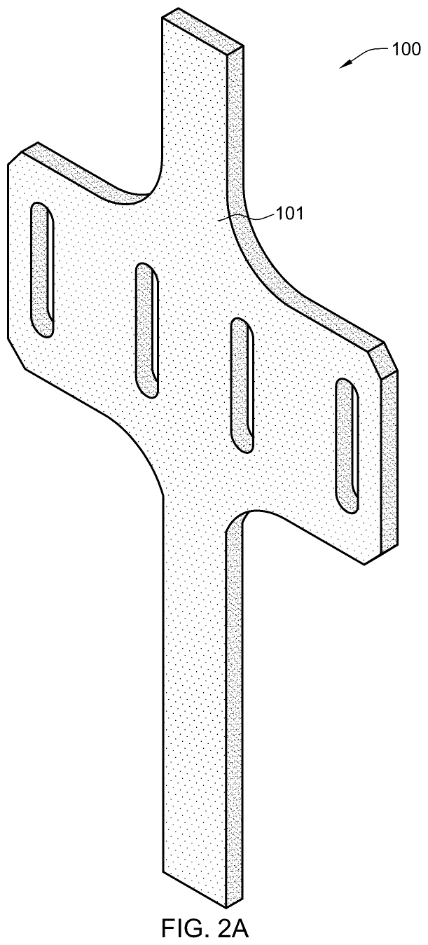

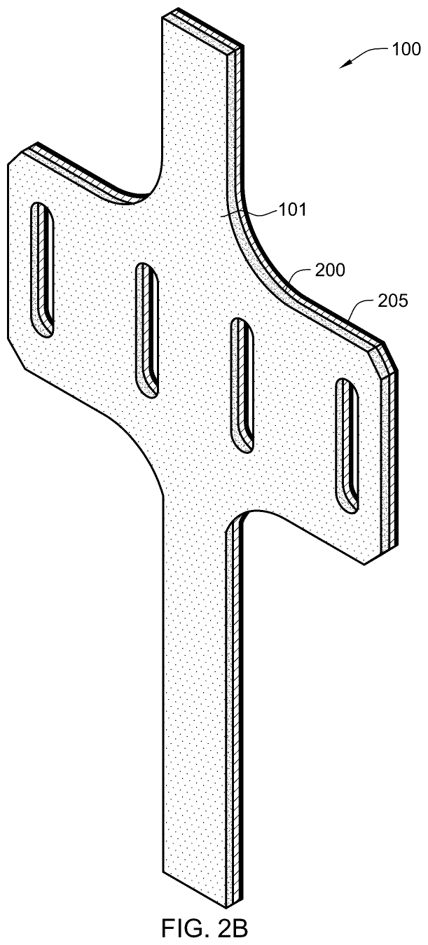

FIGS. 2A and 2B depict isometric views of the frame 100, in accordance with one embodiment of the present invention. The frame 100 is made from a flexible and light weight material, but not limited to polyethylene, polyethylene terephthalate, high-density polyethylene, polypropylene, polystyrene, polyvinyl chloride, polyurethane, poly carbonate, polybutylene terephthalate, acrylonitrile styrene acrylate, acrylics, aluminum, steel, cooper, various other metals, a combination of plastics and metals, or the like. The frame may be comprised of a single layer of material (FIG. 2A) or may be comprised of multiple layers of material (FIG. 2B) to accommodate various design parameters. In one embodiment, the frame 100 is shown with a padding layer 200 which may be made of foam or other open or closed cell materials to provide padding or cushioning material for the wearer to provide a more comfortable fit and a third layer of fabric 205 to minimize chaffing. The thickness of the frame 101 and the thickness of the padding layer 200 are based on the size of the drainage bag 300, and/or the strength of materials used for the frame 101 and the padding layer 200. In some embodiments (FIG. 2B) there is a third layer of material 205 to minimize chaffing. In additional embodiments, multiple additional layers can be added based on design requirements.

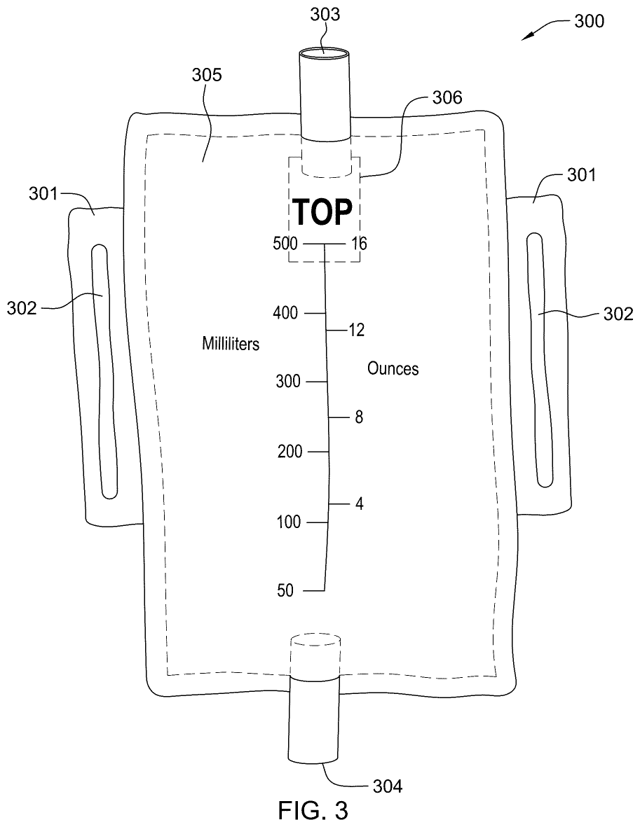

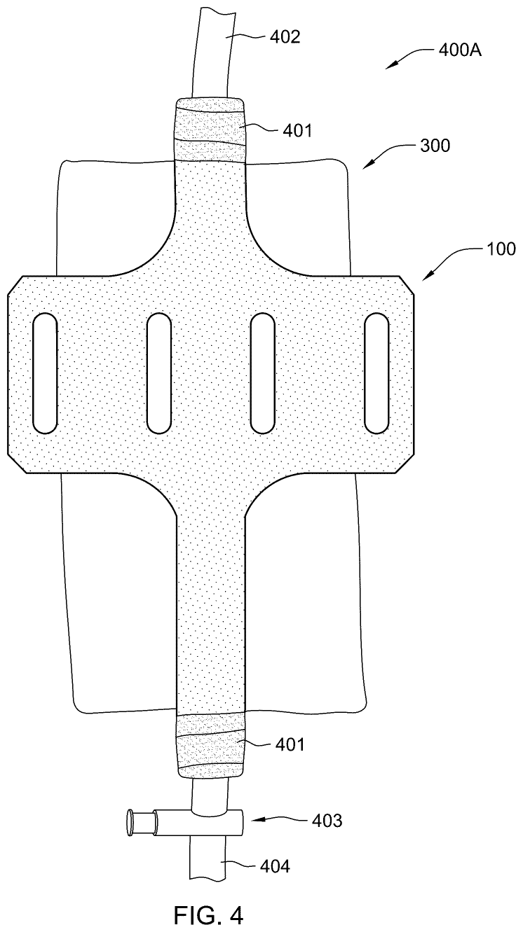

FIG. 3 depicts a front view of a drainage bag, 300 in accordance with one embodiment of the present invention. The drainage bag 300 is shown for exemplary purposes and does not limit the type of drainage bag 300 which can be used with the device. The drainage bag 300 has a main compartment 305 where an inlet stem 303 feeds into a flutter valve 306, that feeds into the main compartment 305. An outlet stem 304 connects to a valve 403 (shown in FIG. 4). In some embodiments, the valve 403 is integrated into the outlet stem 304. In other embodiments, the valve 403 is removable and replaceable. In some embodiments, integrated into both sides of the drainage bag 300 are tabs 301, wherein each tab 301 has a slot 302 which is substantially similar in size and placement to the slots 112 of the frame 100. In these embodiments, the additional tabs 301 allowing for a 4-point attachment to the frame 100 instead of a 2-point attachment utilizing mounting points only at the inlet and outlet stems. The capacity of the drainage bag 300 is based on a variety of factors but is otherwise able to be different sizes.

FIG. 4 depicts a rear view of the 2-point attached urine collection device assembly 400A, in accordance with one embodiment of the present invention. Frame 100 is shown placed behind the drainage bag 300. The drainage bag 300 is secured to the frame 100 at the top and the bottom via an adhesive tape 401. In one embodiment, the adhesive tape 401 is self-fusing silicone tape. Other forms of adhesive tape, such as, but not limited to, heat-shrink tubing or cable-ties may be used. In some embodiments, an adhesive may be used to attach the drainage bag 300 to the frame 100, which may be not limited to glue or the like. The aforementioned embodiments provide for essentially permanent attachment of the drainage bag 300 to the frame 100. In some embodiments the adhesive tape 401 is replaced by a resealable or reusable securing means, such as, but not limited to, a hook and loop style fastener or the like. In another embodiment, a quick-connect latching mechanical interface may be integrated into the frame 100 and the drainage bag 300. Such embodiments would allow for separate and expedient replacement or interchangeability of either the drainage bag 300 and the frame 100. In further embodiments, the drainage bag 300 and the tube 402 are an assembly and are both replaceable or interchangeable. The valve 403 is shown attached to the outlet stem 304. In some embodiments, the valve 403 is attached to another portion of tubing 404. The valve 403 may be a variety of valve types or styles designed to allow the wearer to open or close the valve to empty the contents of the drainage bag 300. Catheter tubing 402 is attached to the inlet stem 303 of the drainage bag 300 and is attached to the catheter. In some embodiments, the drainage bag 300 and the stems (303 and 304) as well as the catheter tubing 402 are welded together through various welding techniques.

In some embodiments, the drainage bag 300 has an internal anti-backflow valve 306 where it connects to the catheter tubing 402.

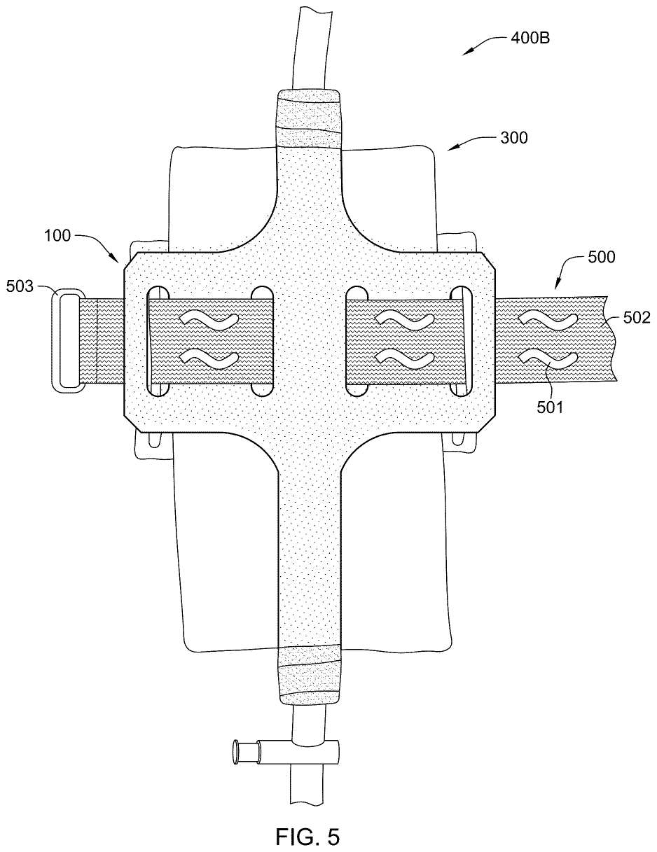

FIG. 5 depicts a rear view of the 4-point attached urine collection device assembly 400B, in accordance with one embodiment of the present invention. The frame 100 is shown attached to drainage bag 300 and a strap 500 is shown attached to both the frame 100 and the drainage bag 300. The strap 500 is woven through slots 112 of the frame and slots 302 of the drainage bag 300. This provides for additional securement means of the drainage bag 300 to the frame 100. In some embodiments, (as shown in FIG. 4) a 2-point attachment of bags lacking side tabs 301, the strap 500 is woven only through the frame 100. The strap 500 is used to secure the frame 100 to the wearer's leg. In the depicted embodiments, the strap 500 is made from an elastic material 502 and has a rubber-like material 501 applied to provide additional support to keep the device in place. The strap 500 is interchangeable and can easily be removed and replaced. The strap 500 has a loop 503 attached to one end.

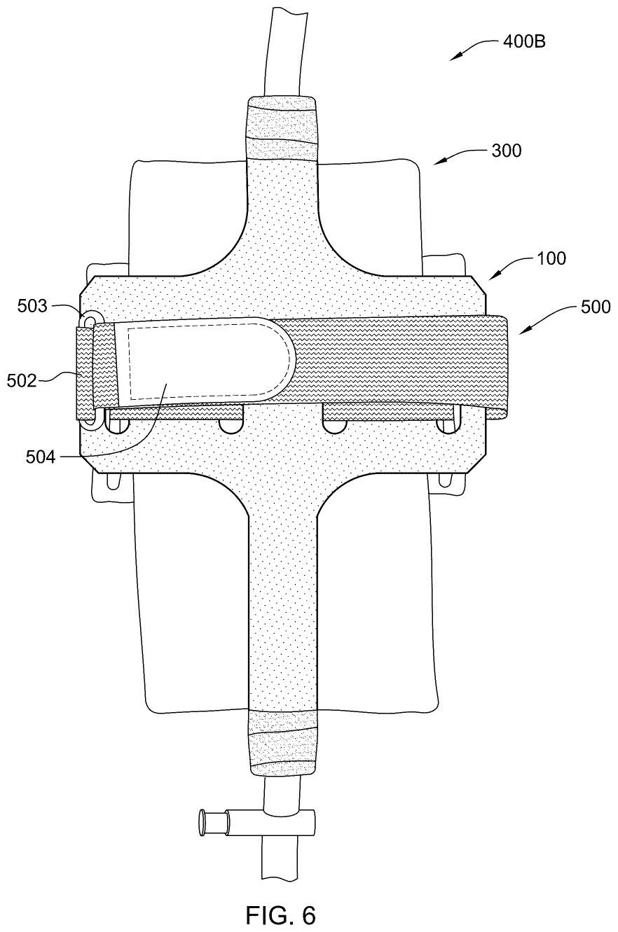

FIG. 6 depicts a rear view of the urine collection device assembly 400B, in accordance with one embodiment of the present invention. The strap 500 is shown in a closed position where the strap 500 passes through the loop 503 and is secured via end tab 504. The strap 500 is secured via a hook and loop style fastener or the like which can be secured and unsecured to allow the wearer to put on, adjust and take off the device.

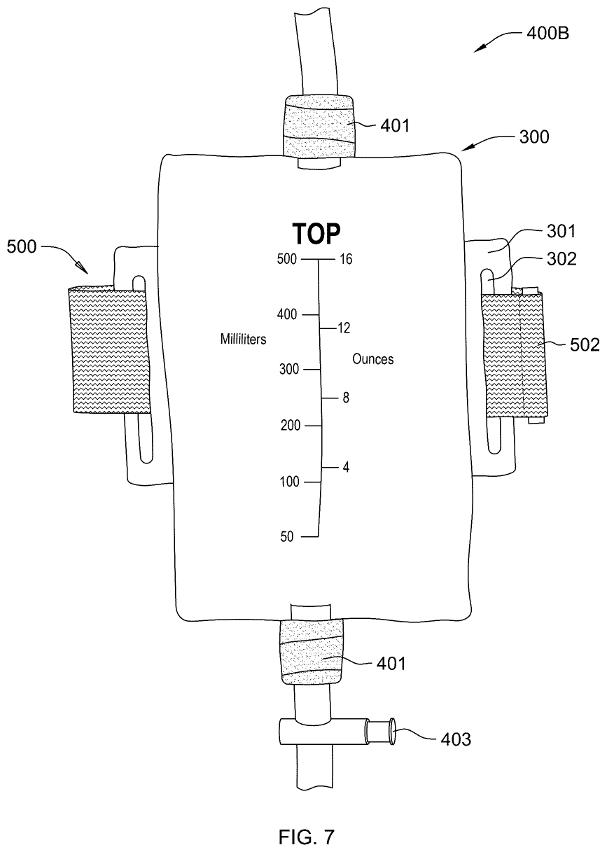

FIG. 7 depicts a front view of the urine collection device assembly 400B, in accordance with one embodiment of the present invention. The strap 500 which passes through slots 302 of the drainage bag 300 in addition to the adhesive tape 401 ensures that the drainage bag 300 is secured to the wearer's leg so that the device is as comfortable and inconspicuous as possible for the wearer. So that the wearer is able to go about their day without any issues or difficulties with the device.

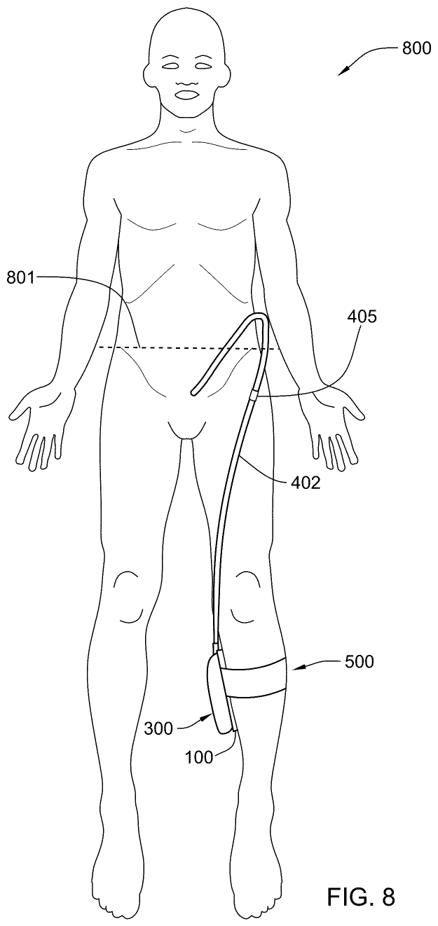

FIG. 8 depicts a view of the urine collection device assembly in use, in accordance with one embodiment of the present invention. A person 800 is shown with the device attached to their leg. The frame 100 and drainage bag 300 are positioned on the interior of the wearer's leg just below the knee. This location is typically of a smaller circumference than the muscular portion of the calf and so assists in preventing the downward movement of the collection system. In the depicted embodiment. the catheter tube 402 utilized in this system is considerably longer (approximately 36 inches) instead of the average 18 inches. The primary reason for this is to accomplish the routing depicted in FIG. 8. As shown, the catheter tube 402 is run from the catheter, up above the waistline 801 and then back down below the waistline 801 and down along the inner thigh and attaching to the drainage bag 300 at the inner side of the upper calf, just below the knee.

This routing provides a number of advantages. The two points where the catheter tube 402 intersects the waistline 801 (belt) are anchored by friction without the use of any adhesive, thereby isolating the catheter connection from any push/pull/tension on the catheter resulting from active motion of the leg and preventing irritation, discomfort, sores and other possible reactions resulting from long-term use of an adhesive anchor patch. The catheter tube 402 passes along the sides of both the hip and knee joints, thereby allowing full articulation of the joints without any prospect for kinking of the tubing and resulting blockage of flow.

The system may include various sensors, transmitters and data logging components or additional devices capable of providing real-time as well as historical perspectives on the user's urinary conditions as well as monitor various physical, chemical and/or biological aspects of the urine. In the depicted embodiment, the various sensors(s) 405 are located in the catheter tube 402. The location within the catheter tube 402 is dependent upon the type of sensor 405 and the type of tubing. In the depicted embodiment, the sensor 405 is located below the waistline 801, but it can also be located above the waistline 801. In some embodiments, based on the sensor type, the sensor 405 is secured around the catheter tube 402. The sensor(s) 405 might include measurement of flow rates, measurement of chemical aspects of the urine, such as but not limited to, pH, salinity, sugar, etc. These sensor(s) 405 may be wholly self-contained in the device or a component of the system, or comprised of connected individual assemblies, such as sensor, transmitter and receiver/data-logger. The sensor(s) 405 and transmitter could be securely located, for example on the frame 100, in the catheter tube 402, or incorporated into the top of the drainage bag 300.

The sensor(s) 405 and/or the transmitters might be fashioned in such a manner as to be disposable or reuseable. The sensor(s) 405 and/or the transmitters are able to transmit the data to a computing device which would be able to process the data to provide specific information to a user to assist them with knowing when the drainage bag is full, chemical irregularities in the urine, a blocked valve, or the like.

Real-time data captured by such a system could be useful in many ways. For example, in prostatectomy patients, the primary one of two sets of muscles used to control flow of urine is removed during surgery. This system, fitted with an inline flowmeter, could be of assistance in training the secondary set of muscles to take primary control of continence. Abundant other data collected by such a system would be useful to patients, caregivers and physicians, limited only by the availability of suitable sensor(s) 405.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

It will be further understood that various changes in the details, materials, and arrangements of the parts which have been described and illustrated in order to explain the nature of this invention may be made by those skilled in the art without departing from the scope of the invention as expressed in the following claims.

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein that are believed as maybe being new, this understanding, as indicated by use of the term “present invention,” is tentative and provisional and subject to change over the course of patent prosecution as relevant information is developed and as the claims are potentially amended.

The foregoing descriptions of various embodiments have been presented only for purposes of illustration and description. They are not intended to be exhaustive or to limit the present invention to the forms disclosed. Accordingly, many modifications and variations of the present invention are possible in light of the above teachings will be apparent to practitioners skilled in the art. Additionally, the above disclosure is not intended to limit the present invention. In the specification and claims the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises”.

Although various representative embodiments of this invention have been described above with a certain degree of particularity, those skilled in the art could make numerous alterations to the disclosed embodiments without departing from the spirit or scope of the inventive subject matter set forth in the specification and claims. Joinder references (e.g., attached, adhered, joined) are to be construed broadly and may include intermediate members between a connection of elements and relative movement between elements. As such, joinder references do not necessarily infer that two elements are directly connected and in fixed relation to each other. Moreover, network connection references are to be construed broadly and may include intermediate members or devices between network connections of elements. As such, network connection references do not necessarily infer that two elements are in direct communication with each other. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements and/or substantial equivalents.

What is claimed is:

1. A urine collection bag mounting system, comprising:
a frame member;
a collection bag secured to the frame; and
a strap, wherein the strap is attached to the frame member.

2. The urine collection bag mounting system of claim 1, wherein the frame member is comprised of one or more layers of material.

3. The urine collection bag mounting system of claim 1, wherein the collection bag has tabs on opposing sides, and at least one slot within the tabs, wherein the strap passes through the slots.

4. The urine collection bag mounting system of claim 1, wherein the collection bag is secured at a top end and a bottom end to the frame.

5. The urine collection bag mounting system of claim 1, wherein the collection bag has a flutter valve at an inlet.

6. The urine collection bag mounting system of claim 1, wherein the collection bag has a valve at an outlet.

7. The urine collection bag mounting system of claim 5, further comprising a tubing attached to the inlet of the collection bag at a first end and attached to a catheter at a second end.

8. The urine collection bag mounting system of claim 1, wherein the frame member has a plurality of slots to receive the strap.

9. The urine collection bag mounting system of claim 1, wherein the strap has a non-slip coating on a first surface, wherein the first surface comes in contact with the wearer's skin.

10. The urine collection bag mounting system of claim 1, further comprising a flow sensor integrated into the collection bag.

11. A urine collection bag mounting system, comprising:
a frame member;
a collection bag having an inlet and an outlet secured to the frame member;
a strap connected to the frame member, wherein the strap is secured around a wearer's leg; and
a tube connected to the inlet of the collection bag.

12. The urine collection bag mounting system of claim 11, wherein the frame member is comprised of at least two layers.

13. The urine collection bag mounting system of claim 12, wherein one of the layers is structural and one layer is padding.

14. The urine collection bag mounting system of claim 11, further comprising a sensor integrated into the collection bag.

15. The urine collection bag mounting system of claim 11, wherein the strap has a non-slip coating.

16. The urine collection bag mounting system of claim 11, further comprising a sensor integrated into the tube.

17. A urine collection bag mounting system, comprising:
A multi-layered frame member;
a collection bag having an inlet and an outlet secured to the frame member;
a strap connected to the multi-layered frame member and the collection bag, wherein the strap is secured around a wearer's leg;
a tube connected to the inlet of the collection bag; and
a sensor in communication with the tube.

18. The urine collection bag mounting system of claim 17, wherein the sensor is wirelessly connected to a computing device.

19. The urine collection bag mounting system of claim 17, wherein the multi-layered frame member has a notched portion of an upper portion and a lower portion.

20. The urine collection bag mounting system of claim 17, wherein the multi-layered frame member has a series of slots where the strap passes through the slots.
